(19) United States
(12) Plant Patent Application Publication
(10) Pub. No.: US 2016/0205836 P1
(43) Pub. Date: Jul. 14, 2016

(54) Peach tree white zest one

(50) ;

(71) Applicant: The Texas A&M University System, College Station, TX (US)

(72) Inventors: David H. Byrne, Bryan, TX (US); Natalie Anderson, Calvert, TX (US)

(73) Assignee: The Texas A&M University System, College Station, TX (US)

(21) Appl. No.: 14/544,503

(22) Filed: Jan. 13, 2015

(51) Int. Cl. A01H 5/00

(52) U.S. Cl. PLT/196

(57) ABSTRACT

Disclosed is a new variety of Prunus persica named ‘White Zest One’. This new variety, which requires approximately 500-550 chilling units of dormancy, is considered to be a peach tree which produces a high quality, sweet-acid, medium large to large, firm, semi freestone to freestone, white-fleshed peach that matures in early to mid-June in the medium chill zone of Texas.

### BACKGROUND OF THE INVENTION

### Field of the Invention

This invention relates to peach trees referred to as a variety of Prunus persica named ‘White Zest One’ ‘White Zest One’, which requires approximately 500-550 chilling units of dormancy, produces a high quality, firm semi freestone to freestone white-fleshed peach which has a sweet acid flavor that matures the early mid-season in the medium chill zone of Texas.

### SUMMARY OF THE INVENTION

The ‘White Zest One’ peach is characterized as to novelty and is otherwise noteworthy by producing fruit that ripens in the early mid-season; is considered high quality; and which is firm and has an attractive coloration. In this regard, the present variety of peach tree bears sweet acid, white-fleshed fruit that are ripe for commercial harvesting and shipment in early to mid-June, when the fruit is grown in the medium chill zone of Texas.

### ORIGIN OF THE VARIETY

The present peach tree was the result of an ongoing Stone Fruit Breeding Program of Texas A & M University, College Station, Brazos County, Tex. To this end, controlled crosses are made each year to produce seedling populations from which improved progenies are evaluated and selected.

The seedling ‘White Zest One’ was originated at the Texas A & M University Horticultural Farm in College Station, Tex. in 2006, and was chosen from a population of seedlings that resulted from seed from a cross between TX3D45W a white fleshed seedling and ‘Tropicprince’ (Tropicpeachone, U.S. Plant Pat. No. 1,296). The parents of TX3D45W are ‘TexRoyal’ (Byrne and Bacon. 1991. HortScience 26:1338-1340) and a white-fleshed seedling of complex heritage with ‘OHenry’, ‘Giant Babcock’, ‘Redwing’ and ‘Red Delight’ in its background. Resulting seed from this cross were planted in 2004 at the Texas A & M University Horticultural Farm in College Station, Tex. The seedling designated as TX3C331W was marked for subsequent observation and noted as having exceptional characteristics. Two-year and older trees of the selection were subsequently evaluated during the 2008 through 2013 fruit growing seasons in California (Fowler) and Texas (Fairfield and College Station).

### ASEXUAL REPRODUCTION OF THE VARIETY

‘White Zest One’ was bud grafted onto virus-free Nemaguard (“The Brooks and Olmo Register of Fruit and Nut Varieties,” 3rd Ed., American Society of Horticultural Science Press, Alexandria, Va., 1997) peach rootstock in June 2006 at the nursery site in Oakdale, Calif. The variety was subsequently planted at the experimental orchard in the central portion of the San Joaquin Valley, near Fowler, Fresno County, Calif. and in three sites in Texas (College Station, Terrell and Fairfield). Fruit from the resulting propagation has been evaluated during the period from 2008 to 2013 fruit seasons. This evaluation clearly demonstrated that the re-propagated trees were true to the characteristics of the original seedling in all observable aspects.

### BRIEF DESCRIPTION OF THE DRAWINGS

This new variety of peach tree is illustrated by the accompanying photographic drawings and depicts the plant by the best possible color representation:

FIG. 1. Fruit of ‘White Zest One’ showing the semi freestone to freestone trait, some red coloration in the white flesh, and excellent red blush and fruit shape. The fruit was harvested from a tree in the experimental orchard in Fairfield, Tex.

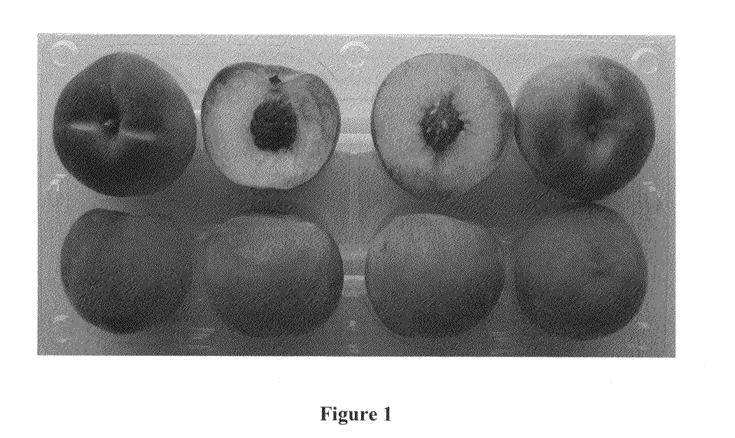

FIG. 2. Color photograph of ‘White Zest One’ fruit showing internal flesh color, shape, and external coloring. The fruit was harvested in the research plots in Fowler, Calif.

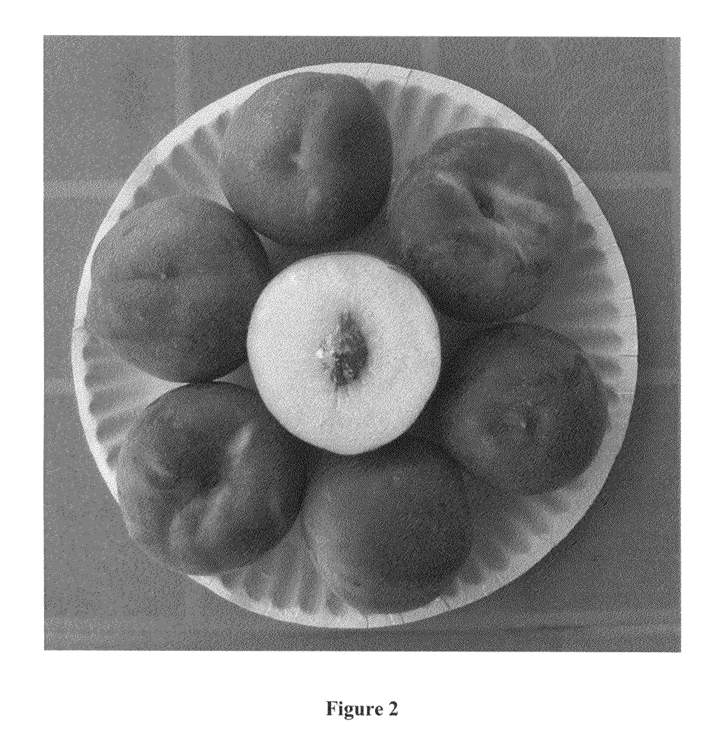

FIG. 3. Color photograph of the endocarp of ‘White Zest One’. The ruler is demarcated in millimeters.

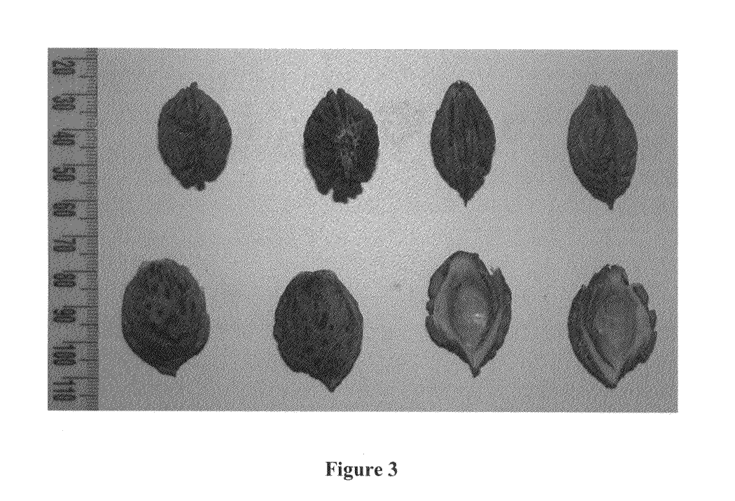

FIG. 4. A stem showing the leaves of the ‘White Zest One’ peach. The ruler is demarcated in millimeters.

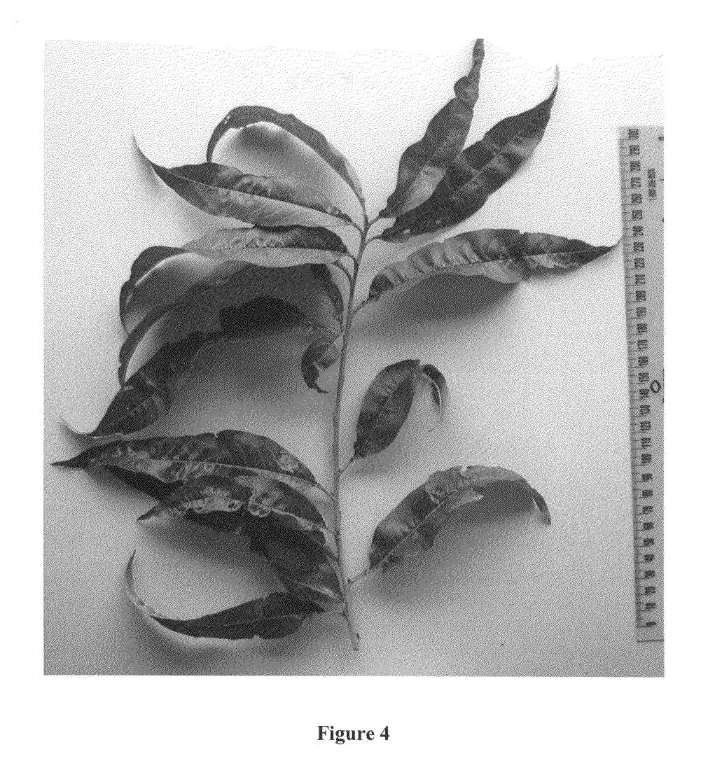

FIG. 5. Showy flowers of ‘White Zest One’. The ruler is in millimeters.

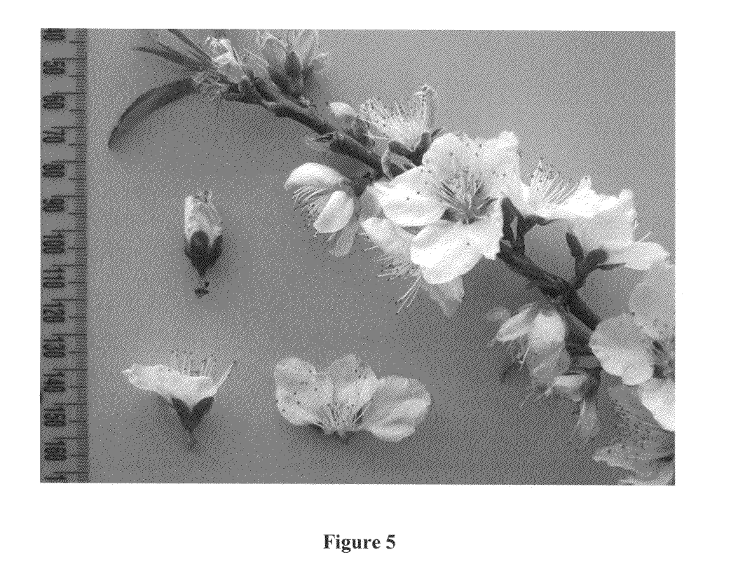

### BOTANICAL DESCRIPTION OF THE VARIETY

Referring more specifically to the pomological details of this new and distinct variety of peach tree, the following has been observed under the ecological conditions prevailing at the experimental orchards in the medium chill zone of Texas (College Station and Fairfield). All major color code designations are by reference to the R.H.S. Colour Chart (Third Edition) provided by The Royal Horticultural Society of Great Britain. Colors are approximate as color depends on horticultural practices such as light level and fertilization rate, among others.
Tree:
   Size.—Generally average to above average as compared to other common peach cultivars ripening in the early mid-season of maturity.
   Height.—11 feet (3.35 m) at the end of the 2013 growing season.
   Width.—14 feet width (4.27 m) at the end of the 2013 growing season.
   Vigor.—High.
   Density.—Medium to high.
   Productivity.—Productive.
   Shape.—The trees are vigorous with the typical semi-spreading growth habit similar to ‘TexKing’, ‘TexPrince’, and ‘TexRoyal’.
   Current season growth.—The current season growth for the new variety was approximately 4.0 to 4.4 feet (1.22-1.34 m).
   Regularity of bearing.—Regular, and considered hardy under typical conditions experienced in the medium chill zone of central Texas (College Station, Fairfield, Terrell) and in central San Joaquin Valley in California.
Trunk:
   Size.—Approximately 6.0 inches (15.2 cm) in diameter and 20.0 inches (50.8 cm) in circumference when measured at a distance of approximately 12 inches (30.5 cm) above the soil level, at the end of the 2013 growing season on a seven-year old tree.
   Bark texture.—Considered moderately rough with numerous folds of papery scarf-like skin being present.
   Bark coloration.—Variable, colors present are 156A-D of the Greyed-White Group, N200C-D of the Brown group.
Branches:
   Size.—Considered medium for the variety.
   Thickness.—Average (about 10 cm in diameter as measured 10 cm from the trunk on a seven-year old tree) as compared to other varieties.
   Surface texture.—Average and appearing furrowed on wood that is several years old.
   Lenticels.—Numerous flat, oval lenticels present. The lenticels range in size from approximately 3 to 7 mm in width and were approximately 1 to 2 mm in height.
   Current season shoots.—Surface texture — Substantially glabrous.
   Internode length.—Approximately 2.0 to 3.0 cm as measured in the middle of a current season stem.
   Color of mature branches.—The predominant colors are 165A and 166A-B of the Greyed-Orange Group, 197A-D and 198A-D of the Greyed-Green Group and N200C-D of the Brown Group.
   Current season shoots.—Color — Light green (143B-C of the Green Group and 144C-D of the Yellow-Green Group) with some reddish-brown coloration appearing on exposed surface of the shoots (166B and 176C-D of the Greyed-Orange Groups). The color of new shoot tips is considered a bright and shiny green (mainly Yellow-Green Groups 144D and 145A-B).
Leaves:
   Size.—Considered moderately large for the species. Leaf measurements have been taken from vigorous upright current season growth approximately at mid-shoot.
   Leaf length.—Approximately 148 to 186 mm.
   Leaf width.—Approximately 35 to 43 mm.
   Leaf thickness.—Less than 1 mm.
   Leaf form.—Lanceolate.
   Leaf tip form.—Acuminate.
   Leaf upper surface color.—Green, approximately 137A-C of the Green Group.
   Leaf lower surface color.—Green, approximately 137C, 138B of the Green Group and 146A, and 147B of the Yellow-Green Group.
   Leaf mid-vein color.—Light green, approximately 145C-D of the Yellow-Green Group.
   Leaf margins.—.
   Form.—Considered crenulate.
   Uniformity.—Considered generally uniform.
   Leaf petioles.—.
   Size.—Considered medium long.
   Length.—Approximately 12 to 14 mm.
   Thickness.—Approximately 1.5 to 2 mm.
   Color.—Pale green (Yellow-Green Group 145C-D).
   Leaf glands.—.
   Size.—Approximately 2-4 mm in height and 1 mm or less in width.
   Number.—Generally 2-3 per leaf.
   Type.—Reniform.
   Color.—Light brown (165C-D and 177B-D of the Greyed-Orange Group).
   Leaf stipules.—.
   Size.—Medium for the species.
   Length.—Approximately 10 to 15 mm.
   Form.—Lanceolate.
   Color.—Green (Yellow-Green 144B-C) with reddish brown tips (Greyed-Orange Groups 165C and 17C-D) when young. The stipules are considered to be early deciduous.
   Ratio of wood (leaf) buds to flowering buds.—1 to 2 flower buds per vegetative bud.
Flowers:
   Floral buds.—.
   General.—The floral buds are considered to be medium to medium large in size, conic in form, and slightly appressed relative to the bearing shoot.
   Color.—The bud scales are grayed-green, (approximately Greyed-White Group 156A-C, Green-White Group 157A-B and the Greyed-Yellow Group 160C). The buds are considered hardy under the typical climatic conditions found in the medium chill zone of Texas and the central San Joaquin Valley, California.
   Length.—Approximately 6 to 8 mm
   Blooming type.—Considered relatively early in relation to other peach cultivars grown in the medium chill zone of Texas. Date of full bloom was between February 20th and March10 th during the period between 2008 and 2013. The average bloom was March 2nd during this time period about 4 days before ‘June Gold’ and about two weeks before ‘Scarlet Pearl’.
   Flower type.—Showy.
   Flower size.—Flower diameter at full bloom is approximately 39 to 46 mm.
   Bloom quantity.—Considered abundant.
   Flower bud frequency.—Normally 1 to 2 per node.
   Petal size.—
   General.—Considered medium large to large for the species.
   Width.—Approximately 10 to 15 mm.
   Length.—Approximately 18 to 24 mm.
   Petal form.—Broadly ovate.
   Petal count.—Five.
   Petal color.—Light pink when young (Red-Purple Group 62C-D, 65C-D and 69A-B), becoming darker near the petal claw.
   Petal claw.—.
   Form.—The claw is considered truncate in shape and has an average size when compared to other varieties.
   Length.—Approximately 1 to 2 mm.
   Width.—Approximately 1 mm.
   Petal margins.—Generally considered variable, from nearly smooth to slightly undulate.
   Petal apex.—Generally — The petal apices appear slightly domed.
   Flower pedicel.—.
   Length.—Present and having an average length of approximately 2 to 4 mm.
   Thickness.—Considered average, approximately 1 mm.
   Color.—A light green (Yellow-Green Group N144B-D and 144B-C).
   Floral nectaries.—.
   Color.—Variable, from medium yellow-green to yellow-orange (Yellow Group 5A-B, 6A-C, 7A-D, 8A-C, 13A-B, Yellow-Orange Group 14A-C, 15A-C, 16A-B and Greyed-Yellow Group 162A-B).
   Calyx.—.
   Surface texture.—Generally glabrous.
   Color.—A brownish red to maroon with green (Yellow-Green Group N144B-D, 144B-C, Greyed-Orange Group 176A-B, Greyed-Purple Group 183A-C, 184A-B and 185A).
   Sepals.—
   Surface texture.—The surface has a short, fine, wooly and a gray-colored texture.
   Size.—Average, and ovate in form.
   Color.—A dull red to maroon with green (Yellow-Green Group N144B-D, 144B-C, Greyed-Orange Group 176A-B, Greyed-Purple Group 183A-C, 184A-B and 185A).
   Anthers.—.
   General.—Moderately long for the species.
   Color.—Golden yellow (Yellow-Orange Group 14A-B, 15A, 16A and 17A-B).
   Pollen production.—Pollen is abundant, and is a yellow color.
   Filaments.—Size — Variable in length, approximately 16 to 18 mm, with the filaments equal to or slightly longer than the pistil.
   Color.—White when young (approximately White Group 155A-D and N155B-C) and darkening to light pink (Red-Purple Group 62C-D, 65B-D and 69A-B) with advanced maturity.
   Pistil.—.
   General.—Average in size, but equal to or slightly shorter to the general anther height.
   Length.—Approximately 19 to 21 mm, including the ovary.
   Color.—Considered a very light yellow-green when young (Green-Yellow Group 1B-D and Yellow-Green Group 151C), and becoming slightly darker with advancing senescence.
   Surface texture.—The variety has a long, silver white pubescent pistil (approximately White Group 155A-D).
Fruit:
   Maturity when described.—The present variety of fruit is described, as it would be found in its firm ripe condition at full commercial maturity. Under the ecological conditions prevailing in the medium chill of Texas this cultivar is picked from early June until mid June depending on the year and the site of the orchard. The average time of harvest for Fairfield, Tex. was June 8th which was about 10 days after ‘June Gold’ and 7-9 days before ‘Scarlet Pearl’ and White Delight Two’.
   Size.—General — Medium large to large for the season and considered uniform.
   Average cheek diameter.—Approximately 60 to 64 mm.
   Average suture diameter.—Approximately 63 to 65 mm.
   Average axial diameter.—Approximately 55 to 56 mm.
   Fruit form.—Generally considered round to oblate. The fruit is generally uniform in symmetry when viewed from the apical aspect.
   Fruit suture.—Generally, the suture appears as a thin line that extends from the base to the apex, and appears deeper at the apex, forming a shallow basin at the apical point. No apparent callusing or stitching exists along the suture line.
   Color.—Medium red (Red Group 46B) plus all colors of the blush and underlying ground color.
   Ventral surface.—Form — Considered uniform.
   Stem cavity.—Size — Considered average for the species.
   Width.—Approximately 15 to 25 mm.
   Length.—Approximately 23 to 25 mm.
   Depth.—Approximately 13 to 16 mm.
   Fruit base.—Flat.
   Fruit apex.—Flat.
   Fruit stem.—Length — 10-11 mm Thickness — 5-6 mm Color — Medium to light green (Yellow-Green Group 144A-C).
   Fruit skin.—Generally considered medium or average in thickness. Surface Texture — thick pubescence. Skin Acidity — Considered neutral. Tenacious to Flesh — Yes at commercial maturity. Tendency to Crack — Not observed. Skin Color — Generally — Variable, with approximately 60-80% of the fruit surface covered with an attractive orange red blush. Down — Thick and Long. Blush Color — The blush color is generally more prevailing apically. This blush ranges from a medium red (Red 39A) to red (Red 42A) with many degrees of shading and blending occurring between these colorations. Skin Ground Color — Pale yellow (Yellow Group 4D).
   Flesh color.—Ivory (White Group 155C).
   Flesh fibers.—Present, numerous and lightly colored. These fibers are present throughout the flesh.
   Stone cavity color.—Medium red (Orange-Red Group 34A).
   Flesh texture.—Generally, the flesh is considered firm at commercial maturity.
   Ripening.—Generally the fruit of the present variety ripens evenly.
   Flavor.—Considered sweet, with a slightly acidic flavor.
   Aroma.—Pleasant and reasonably abundant.
   Eating.—Generally considered very good to excellent, particularly for an early mid-season ripening variety.
Stone:
   Attachment.—Freestone at commercial maturity.
   Stone size.—Generally considered medium to medium-large relative to the ratio of stone to fruit size.
   Length.—Approximately 32 to 33 mm.
   Width.—Approximately 25 to 27 mm.
   Thickness.—Approximately 18 to 20 mm.
   Fibers.—Generally a few medium length fibers are attached sporadically along the surface of the stone.
   Stone form.—Round.
   Stone base.—Wide.
   Apex shape.—The stone apex is wide.
   Stone shape.—Considered variable. The stone is normally ovoid to elongated.
   Stone surface.—.
   Surface texture.—Minor surface markings are honeycombed with numerous single pits and chains of pits.
   Ridges.—Numerous fine ridges are present basally, and converge towards the base of the stone.
   Ventral edge.—Small.
   Dorsal edge.—Shape — Grooved and having moderately rough edges.
   Stone color.—The color of the dry stone is a medium to dark brown (Greyed-Orange Groups 164A, 165A-B and 166A and Greyed-Brown Group N199B-D.
   Tendency to split.—Splitting was not observed.
   Kernel.—the kernel fills the endocarp at harvest and measures approximately 4-5 mm in thickness, 11-12 mm in width, and 15-17 mm in length. When dried the shriveled kernels measure approximately 3-4 mm in thickness, 11-12 mm in width, and 15-16 mm in length. The colors of the shriveled kernels are primarily Greyed-Orange Group 165B-D.
   Use.—The subject variety, ‘White Zest One’, is considered to be a peach tree of early mid-season maturity, which produces fruit which are firm, attractively colored, white-fleshed, with an sweet-acid flavor and which are useful for both local and regional fresh fruit markets.
   Keeping quality.—Good.
   Resistance to insects and disease.—No particular susceptibilities were noted.
   Shipping quality.—Average. Although the new variety of peach tree possesses the described characteristics when grown under the ecological conditions prevailing near College Station and Fairfield, Tex., it will be understood that variations of the usual magnitude and characteristics incident to the changes in growing conditions, fertilization, pruning, and pest control are to be expected.

We claim:

1. A new and distinct Prunus persica tree, substantially as illustrated and described herein.
